SEQUENCE LISTING (8 sequences)

SEQ ID NO: 1 - DNA, 1979 bp, Artificial Sequence
  A synthetic oligonucleotide
gctccgaattcgcccttgacagcagagatccagtttggttaattaattggctccggtgcccgtcagtgggcagagcgcacatcgcccacagtccccgagaagttggtgggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaagggcgaattcgacccaagtttgtacaaaaaagcagatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattga

SEQ ID NO: 2 - DNA, 1985 bp, Artificial Sequence
  A synthetic oligonucleotide
gctccgaattcgcccttgacagcagagatccagtttggttaattaattggctccggtgcccgtcagtgggcagagcgcacatcgcccacagtccccgagaagttggtgggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaagggcgaattcgacccaagtttgtacaaaaaagcagatggtgagcaagggcgaggagctgttcaccggggtggtgcccatcctggtcgagctggacggcgacgtaaacggccacaagttcagcgtgaggggcgagggcgagggcgatgccaccaacggcaagctgaccctgaagttcatctgcaccaccggcaagctgcccgtgccctggcccaccctcgtgaccaccctgagccacggcgtgcagtgcttcgcccgctaccccgaccacatgaagcagcacgacttcttcaagtccgccatgcccgaaggctacgtccaggagcgcaccatcttcttcaaggacgacggcacctacaagacccgcgccgaggtgaagttcgagggcgacaccctagtgaaccgcatcgagctgaagggcgtcgacttcaaggaggacggcaacatcctggggcacaagctggagtacaacttcaacagccacaacatctatatcatggccgtcaagcagaagaacggcatcaaggtgaacttcaagatccgccacaacgtggaggacggcagcgtgcagctcgccgaccactaccagcagaacacccccatcggcgacggccccgtgctgctgcccgacagccactacctgagcacccagtccgtgctgagcaaagaccccaacgagaagcgcgatcacatggtcctgctggagttccgcaccgccgccgggatcactctcggcatggacgagctgtacaagtaa

SEQ ID NO: 3 - DNA, 720 bp, Artificial Sequence
  A synthetic oligonucleotide
atggtgagcaagggcgaggagctgttcaccggggtggtgcccatcctggtcgagctggacggcgacgtaaacggccacaagttcagcgtgaggggcgagggcgagggcgatgccaccaacggcaagctgaccctgaagttcatctgcaccaccggcaagctgcccgtgccctggcccaccctcgtgaccaccctgagccacggcgtgcagtgcttcgcccgctaccccgaccacatgaagcagcacgacttcttcaagtccgccatgcccgaaggctacgtccaggagcgcaccatcttcttcaaggacgacggcacctacaagacccgcgccgaggtgaagttcgagggcgacaccctagtgaaccgcatcgagctgaagggcgtcgacttcaaggaggacggcaacatcctggggcacaagctggagtacaacttcaacagccacaacatctatatcatggccgtcaagcagaagaacggcatcaaggtgaacttcaagatccgccacaacgtggaggacggcagcgtgcagctcgccgaccactaccagcagaacacccccatcggcgacggccccgtgctgctgcccgacagccactacctgagcacccagtccgtgctgagcaaagaccccaacgagaagcgcgatcacatggtcctgctggagttccgcaccgccgccgggatcactctcggcatggacgagctgtacaagtaa

SEQ ID NO: 4 - DNA, 714 bp, Artificial Sequence
  A synthetic oligonucleotide
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattga

SEQ ID NO: 5 - DNA, 1275 bp, Artificial Sequence
  A synthetic oligonucleotide
tggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaagctagaattgtgagcggataacaattccacagtcgaccctaggttgtgtcgcgagtgttggatcccagctgacaccaattgtgagcgctcacaattgctagccgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggt

SEQ ID NO: 6 - DNA, 220 bp, Artificial Sequence
  A synthetic oligonucleotide
gctccgaattgggacagcagagatccagtttggttaattaatagacggagtactgtcctccgagcggagtactgtcctccgactcgagcggagtactgtcctccgatcggagtactgtcctccgcgaattccggagtactgtcctccgaagacgctagcggggggctataaaagggggtgggggcgttcgtcctcactctcaattcggcccaattcgacc

SEQ ID NO: 7 - DNA, 338 bp, Artificial Sequence
  A synthetic oligonucleotide
aattgtgagcgctcacaattgcaggtccgaggttctagacgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcaattgtgagcgctcacaatt

SEQ ID NO: 8 - DNA, 1243 bp, Artificial Sequence
  A synthetic oligonucleotide
gctccgaattcgcccttgacagcagagatccagtttggttaattaattggctccggtgcccgtcagtgggcagagcgcacatcgcccacagtccccgagaagttggtgggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaagggcgaattcgac

(19) United States
(12) Patent Application Publication
(10) Pub. No.: US 2013/0137604 A1
(43) Pub. Date: May 30, 2013

(54) METHODS OF EVALUATING GENETIC ELEMENTS

(71) Applicant: Raytheon BBN Technologies Corp., Cambridge, MA (US)

(72) Inventors: Aaron Daniel Adler, Sharon, MA (US); Jacob Stuart Michael Beal, Somerville, MA (US); Fusun Yaman-Sirin, Arlington, MA (US)

(73) Assignee: Raytheon BBN Technologies Corp., Cambridge, MA (US)

(21) Appl. No.: 13/689,415

(22) Filed: Nov. 29, 2012

Related U.S. Application Data
(60) Provisional application No. 61/565,434, filed on Nov. 30, 2011.

(51) Int. Cl. C12Q 1/68

(52) U.S. Cl. CPC C12Q 1/6897
USPC 506/16; 435/6.1; 435/320.1; 506/26; 435/254.2; 435/254.11; 435/348; 435/410; 435/325; 435/252.33; 435/252.31; 435/369; 435/358; 435/357; 435/367; 435/365

(57) ABSTRACT

The invention relates to compositions for evaluating a genetic element and methods of use.

### PRIORITY APPLICATION(S)

This application claims the benefit of priority under 35 U.S.C. §119(e) to U.S. Provisional Application No. 61,565,434, filed Nov. 30, 2011, entitled “Methods of Evaluating Genetic Elements,” which is incorporated herein by reference in its entirety.

### BACKGROUND OF THE INVENTION

A quantitative understanding of living cells will require methods to perturb and control the activities of their constituent proteins at fine spatial and temporal resolutions. By measuring responses to precise perturbations, predictive models of genetic and cellular networks can be tested and iteratively improved.

Genetic circuits provide a method to design and control perturbations, which contributes to the development of predictive models of cellular networks. Genetic circuits have been built that encode functions that are analogous to electronic circuits, and genetic programs have been built by combining multiple circuits. For example, an “edge-detector” program created bacteria that were able to detect the edge between light and dark regions of an image projected on a plate (Salis et al., Molec. Systems Biol. 6:1-11, 2009). An understanding of the activity of genetic elements is important for the design and development of such circuits.

### SUMMARY OF THE INVENTION

The present invention is based, at least in part, on the discovery of a new method for determining activity of a DNA promoter in vivo (i.e., inside of a living cell). This can be used to speed the development of engineered biological systems by allowing a practitioner to inexpensively and easily monitor the behavior of different pieces of a system at different times and on a large scale (e.g., measurement of tens of thousands of individual cells). Conventionally, testing of a genetic circuit requires that a DNA variation of the circuit be constructed to measure the activity of particular circuit elements, such as DNA promoters. This process is time-consuming and error-prone since it may not be possible to guarantee that the modified test circuit is equivalent to the original circuit. In contrast, aspects and embodiments of the present invention allow monitoring of circuit elements in vivo using “probe” plasmids, as discussed further below.

In some embodiments, a method for debugging genetic circuit systems in vivo is provided. Under current practices, debugging a system typically requires building large plasmids containing multiple devices. This process often takes weeks, particularly as larger plasmids are more prone to experimental problems. If the circuit does not behave as predicted, then the expression levels of intermediate products often cannot be tested without spending weeks on building variant plasmids with different arrangements of fluorescent proteins or other reporters.

Large plasmid systems are often necessary in complex genetic circuits. To debug such systems without the construction of variations with various reporters added, which is time consuming and may not produce equivalent results, a novel “probe plasmid” technique is provided herein. In some embodiments, a probe plasmid contains two different colored fluorescent proteins, one constitutively produced and one regulated by a promoter to be probed. By inserting an appropriate probe plasmid into cells and applying analytic techniques, the operation of any device can be measured in vivo without modification of the plasmid system. The probe plasmids described herein speed debugging by allowing in vivo measurement of intermediate products in a circuit without modification of the circuit.

In some systems, multiple species of plasmid are co-transfected or co-transformed. Using characterization techniques the operation of a system can be tested with the devices split over many easier-to-construct, simpler plasmids, yet still have predictive power for when many parts are on the same plasmid. This also removes major confounding factors of interaction between parts caused by their adjacency on a plasmid, as well as the silencing that often occurs in systems integrated into the chromosome of mammalian cells.

Characterization techniques useful for analyzing the probe plasmid data are well known to those of skill in the art. In some embodiments the characterization techniques provide high-precision single cell analysis. In other embodiments, the characterization techniques focus on population level analysis. Characterization techniques include but are not limited to, fluorescence activated cell sorting (FACS), microscopy, fluorometry, immunohistochemistry, and mass spectrometry.

In some embodiments, skilled users may investigate individual parts of the circuit by choosing appropriate probe plasmids. In other embodiments, such as for non-traditional users, automatically-generated probe plasmids can be used to debug and/or monitor a particular aspect of the genetic circuit.

In one embodiment, a candidate genetic element (e.g., DNA promoter) which is the subject of evaluation, is coupled with a genetic element having known properties, e.g., a DNA promoter having a known strength. The candidate genetic element (also called a test genetic element) controls expression of a first reporter molecule (also called herein a “test reporter molecule,” e.g., a “test reporter protein”), and the genetic element of known activity (called a control genetic element) controls expression of a second reporter molecule (also called herein “a control reporter molecule,” e.g., a “control reporter protein”). The test element and the test reporter molecule (e.g., fluorescent protein), and the control element and the control reporter molecule are on a single plasmid or are on two different plasmids. Expression of the control reporter molecule in comparison to the strength of expression of the test reporter molecule allows the activity of the test genetic element (e.g., the strength of the test promoter) to be calibrated on a per cell basis.

In embodiments where the test element and the test reporter molecule are on a single plasmid, these components may be operably linked. “Operably linked” as used herein means that the test reporter is linked to the candidate genetic element (DNA promoter) in a manner that allows for expression of the reporter.

In one aspect, the invention features a method of evaluating a genetic element by providing a cell (e.g., a mammalian cell or a bacterial cell) comprising a nucleic acid molecule (e.g., a plasmid) where the nucleic acid molecules comprise a test genetic element that controls expression of a first reporter protein, and a control genetic element that controls expression of a second reporter protein. The cell is incubated for a time and under conditions sufficient to allow expression of the test genetic element; and the relation of expression of the first reporter gene to the second reporter gene is measured. In one embodiment, the step of providing a cell comprising a nucleic acid molecule comprising a test genetic element that controls expression of a first reporter protein, and a control genetic element that controls expression of a second reporter promoter comprises (i) providing the nucleic acid molecule comprising a test genetic element that controls expression of a first reporter gene, and a control genetic element that controls expression of a second reporter gene, and (ii) transfecting the nucleic acid molecule into the cell.

In another embodiment, the genetic element is a DNA promoter, enhancer, repressor or upstream or downstream regulatory sequence. In another embodiment the genetic element is a constitutive promoter, an inducible promoter, an activatable promoter, a repressible promoter or a hybrid promoter.

In some embodiments, the first and second fluorescent proteins have minimal spectral overlap. For example, in one embodiment, the first reporter gene and the second reporter gene encode fluorescent proteins. For example, in one embodiment, the first reporter protein is Enhanced Blue Fluorescent Protein 2 (EBFP2), mKate, infrared fluorescent protein (iRFP) or enhanced yellow fluorescent protein (EYFP), and the second fluorescent protein is ERFP2, mKate, iRFP or EYFP, and the second reporter gene is different than the first reporter gene. In one embodiment, the first reporter gene and the second reporter gene are in a mammalian cell.

In another example, the first reporter gene encodes Green Fluorescent Protein (GFP), Red Fluorescent Protein (RFP) or Blue Fluorescent Protein (BFP), and the second reporter gene encodes GFP, RFP or BFP, and the second reporter gene is different than the first reporter gene. In one embodiment, the first reporter gene and the second reporter gene are in a prokaryotic cell, e.g., an E. coli cell.

In one embodiment, the test genetic element is identical to an element that is in a circuit, or that occurs in a natural biological system.

In one embodiment, a plasmid comprising the test genetic element and the control genetic element and the first and second reporter transcription units does not replicate in mammalian cells, and in another embodiment, the plasmid replicates in bacterial cells, e.g., in E. coli cells. In yet another embodiment, the plasmid replicates in mammalian cells.

In one embodiment, the test genetic element controlling expression of the first reporter gene, and the control genetic element controlling expression of the second reporter gene are positioned in the same orientation on the nucleic acid.

In other embodiments, the cell comprises one copy of the nucleic acid comprising a test genetic element that controls expression of first reporter gene, and a control genetic element that controls expression of a second reporter gene. Stable integration and selection with a selectable marker are contemplated for use in the cell comprising one copy of the nucleic acid.

In one embodiment, the cell comprises multiple copies of the nucleic acid comprising a test genetic element that controls expression of a first reporter gene, and a control genetic element that controls expression of a second reporter gene.

The test genetic element can be a member of a library.

In one aspect, the invention features a circuit comprising a nucleic acid molecule comprising a test genetic element that controls expression of a first reporter gene, and a control genetic element that controls expression of a second reporter gene.

In some embodiments, the genetic element is a DNA promoter, an enhancer element, repressor element or activator sequence, or an upstream or downstream regulatory element. In another embodiment, the first and second reporter genes encode a first and second fluorescent protein, respectively.

In one aspect, the invention features a cell comprising a nucleic acid molecule comprising a test genetic element (e.g., a DNA promoter) that controls expression of a first reporter gene, and a control genetic element that controls expression of a second reporter gene. In some embodiments, the first and the second reporter genes encode fluorescent proteins.

In one aspect, the invention features a method of identifying a genetic element (e.g., a DNA promoter) for use in a circuit, by providing a plurality of test genetic elements, wherein each test genetic element of the plurality is provided in a separate test nucleic acid molecule for control of a first reporter gene, and each test nucleic acid molecule further comprises a control genetic element that controls expression of a second reporter gene. One or more of the test nucleic acid molecules are expressed in a cell for a time and under conditions sufficient to allow expression of the first and second reporter genes from the test genetic element and the control genetic element, respectively; and the relation of expression of the first reporter gene and the second reporter gene is measured. If the relation of expression of the first reporter gene and the second reporter gene is determined to be at a desired level for use in the circuit, then the test genetic element is chosen for use in the circuit.

In one embodiment, the first and the second reporter genes encode fluorescent proteins.

In one aspect, the invention features a library comprising a plurality of test genetic elements (e.g., candidate DNA promoters) where each test genetic element of the plurality is positioned 5′ to a first reporter gene, wherein each first reporter gene is positioned 5′ to a control genetic element (e.g., a control DNA promoter) that is positioned 5′ to a second control reporter gene. A spacer region, optionally comprising an insulator sequence separates the first reporter gene and the control genetic element. In one embodiment, the test genetic element/first reporter gene is on a first plasmid, and the control genetic element/control reporter gene is on a second plasmid.

In one embodiment, each test genetic element of the plurality is flanked on either side by a means for cloning, e.g., a restriction enzyme site or a sequence for recombination cloning.

In another embodiment, the first reporter gene and the second reporter gene encode a first and second reporter protein, respectively.

In one aspect, the invention features a method of making a library comprising a plurality of test genetic elements, where each test genetic element of the plurality is introduced into a plasmid, each test genetic element of the plurality is positioned 5′ to a first reporter gene, and each first reporter gene is positioned 5′ to a control genetic element that is positioned 5′ to a second control reporter gene. In some embodiments, each test genetic element is introduced into the plasmid by a restriction enzyme based method or by a recombination cloning method.

In one embodiment, the test genetic element, and the control element are DNA promoter elements, and in another embodiment, the first reporter gene and the second reporter gene encode a first and second reporter protein, respectively.

Embodiments may be implemented in one or a combination of hardware, firmware and software. Embodiments may also be implemented as instructions stored on a computer-readable storage medium, which may be read and executed by at least one processor to perform the operations described herein. A computer-readable storage medium may include any non-transitory mechanism for storing information in a form readable by a machine (e.g., a computer). For example, a computer-readable storage medium may include read-only memory (ROM), random-access memory (RAM), magnetic disk storage media, optical storage media, flash-memory devices, and other storage devices and media.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described herein. The materials, methods, and examples are illustrative only and not intended to be limiting. Other features of the disclosure are apparent from the following detailed description and the claims.

### DESCRIPTION OF THE DRAWINGS

FIG. 1A is a diagram of a circuit for which monitoring strength of an intermediate promoter would be useful. Rightward pointing arrows represent promoters.

FIG. 1B is an example of the coupling of a test promoter (the left-most arrow) with a control promoter (right-most arrow) for measuring the strength of the test promoter. The test promoter controls expression of a test fluorescent protein (RFP (Red Fluorescent Protein)), and the control promoter controls expression of a second fluorescent protein (EBFP (Enhanced Blue Fluorescent Protein)).

FIG. 2A is a diagram of a circuit that has not performed as expected.

FIG. 2B is a diagram of one method of debugging the failed circuit, wherein the entire circuit is recreated with the inclusion of a fluorescent tag inserted under control of an example of one suspect promoter. The “fluorescent tag” is a gene encoding a fluorescent protein.

FIG. 3 is a diagram of an exemplary test promoter/test fluorescent transcription unit (UAS_{TAL1}) coupled to a constitutive control promoter CAG that regulates expression of a second fluorescent protein, mKATE.

FIG. 4 is a diagram illustrating a method of creating a tester probe plasmid, by cloning a test promoter 5′ to a first fluorescent protein transcription unit and a control promoter/second fluorescent protein transcription unit that are contained on a stock probe plasmid. The test promoter is cloned into by a recombination cloning method using the recombination sequences attB1, attB2, attP1 and att P2.

FIG. 5 is the nucleotide sequence of the constitutive promoter Elena fused to the fluorescent protein gene mKATE (“Hef1a-mKATE”) (BioBricks reference BBA_k511801; SEQ ID NO:1). The mKATE cDNA sequence is underlined.

FIG. 6 is the nucleotide sequence of the constitutive promoter Hef1a fused to the fluorescent protein gene EBFP2 (“Hef1a-EBFP2”) (BioBricks reference BBA_k511800; SEQ ID NO:2). The EBFP2 sequence is underlined.

FIG. 7A is the nucleotide sequence of the Enhanced. Blue Fluorescent Protein 2 (EBFP2) (BioBricks reference BBA_k511100; SEQ ID NO:3).

FIG. 7B is the nucleotide sequence of the red fluorescent protein, mKATE (BioBricks reference BBA_k511102; SEQ ID NO:4).

FIG. 8A is the nucleotide sequence of the Test promoter Hef1A-LacOid (BioBricks reference BBA_k511000; SEQ ID NO:5).

FIG. 8B is the nucleotide sequence of the Test promoter UAS-GAL4 (BioBricks reference BBA_k511003; SEQ ID NO:6).

FIG. 9A is the nucleotide sequence of the Test promoter TRE-Tight-LacOid promoter (BioBricks reference BBA_k511004; SEQ ID NO:7).

FIG. 9B is the nucleotide sequence of the Hef1A promoter (BioBricks reference BBA_k415508; SEQ ID NO:8).

### DETAILED DESCRIPTION OF THE INVENTION

Provided herein is a new method for determining activity of a test genetic element, e.g., a DNA promoter, in vivo (i.e., inside of a living cell). This can be used to speed the development of engineered and natural biological systems by allowing a practitioner to inexpensively and easily monitor the behavior of different pieces of a design at different times and on a large scale (e.g., measurement of tens of thousands of individual cells). By the new method, a test genetic element and a control genetic element are coupled on the same or different plasmids, and the relative activity of the elements is evaluated to debug, or evaluate, different parts of the circuit. The new methods are useful, for example, for the development and evaluation of artificial circuits and naturally occurring systems in living cells.

The methods featured in the invention allow for the evaluation of a test genetic element in a living cell by coupling the test element with a first reporter gene, e.g., a first fluorescent reporter gene, and measuring reporter gene expression relative to that of a second reporter gene that is regulated by a control element, e.g., a genetic element having a known characteristic. The evaluation of the test element is useful at least for the evaluation of the element as a component of a circuit that is engineered in a living system, e.g., in a living cell. The method also provides an assay to evaluate a genetic element, e.g., a DNA part, such as a DNA promoter, in isolation for use in a larger system, such as a circuit.

Circuits

As used herein, a “circuit” is a collection of DNA parts, e.g., transcription units, that encode proteins or protein fragments, such as protein fragments having a particular function. The DNA parts of the circuit interact to activate or repress expression of each protein or protein fragment. In some embodiments, the expression of the circuit will cause the cell to respond in a certain predictable way. For example, expression of a circuit can cause a cell to take on a particular shape or structure, or can cause the cell to respond in a certain way to its environment, e.g., to grow towards light, or away from light.

These synthetic genetic circuits can potentially result in a wide range of productive systems. For example, synthetic genetic circuits have been evaluated for the development of, e.g., more efficient pathways for the production of antimalarial drugs (Ro et al., Nature 440:940-943, 2006), bacteria that can metabolize toxic chemicals (Kim et al., Integr. Biol. (Camb) 3:126-133, 2011, Epub, Aug. 17, 2010), or bacteria that can hunt and kill tumors (Anderson et al., J. Mol. Biol. 355:619-627, 2006, Epub. Nov. 14, 2005).

Evaluation of a Test Genetic Element

As provided by the methods featured in the invention, a test genetic element, such as a promoter can be evaluated. Evaluation of a test genetic element comprises cloning a first reporter protein gene (e.g., transcription unit tier a first fluorescent protein gene) under control of the test genetic element, and cloning a second reporter protein gene (e.g., transcription unit for a second fluorescent protein gene) under control of a control genetic element. In one embodiment, the test genetic element/test reporter protein transcription unit and the control genetic element/control reporter protein transcription unit are cloned onto the same plasmid, and in another embodiment, the test genetic element/test reporter protein transcription unit and the control genetic element/control reporter protein transcription unit are cloned onto separate plasmids.

As used herein, a “genetic element” is a fragment of DNA (deoxyribonucleic acid) or RNA (ribonucleic acid) that contains information that can influence a biological activity in a cell. For example, a genetic element on DNA can influence transcription by containing a binding site for transcriptional regulatory proteins such as transcription factors which can promote or inhibit binding by RNA polymerase and other transcriptional machinery, thereby promoting or inhibiting transcriptional activity. Genetic elements can also influence sites of nucleic acid modification, such as methylation, which can influence chromosome structure, which can influence transcriptional activity. Genetic elements on RNA can also influence binding of RNA binding proteins on the RNA to, for example, block or enhance ribosome binding to inhibit or promote translation. Genetic elements on RNA can also cause premature termination of the RNA polymerase machinery to inhibit production of full-length protein, or the elements can recruit proteins that block splicing events, or enhance splicing events, such as by recruiting splicing machinery. A DNA promoter is one exemplary genetic element.

As used herein, a “device” is any collection of DNA parts that are associated to form a computational unit. For example, a repressor device consists of a repressible promoter and a coding sequence for the repressor protein, along with any other associated control elements such as degradation tags or miRNA.

The methods featured in the invention are particularly suited for evaluating DNA promoters, e.g., the strength of DNA promoters, but one of skill in the art will understand that the methods described herein can be used for the evaluation of any genetic element.

As used herein a “DNA promoter” is a DNA sequence that facilitates transcription of a gene. The promoter is typically located at the 5′ end of the gene (or transcription unit) that the promoter regulates. Promoters contain DNA sequences and response elements that provide an initial binding site for the RNA transcriptional machinery (including RNA polymerase and transcription factors that recruit the RNA polymerase). Transcription factors bind specific activator and repressor sequences that attach to certain promoters and regulate gene expression.

Promoters can be various lengths. As used in the compositions and methods featured in the present invention, a promoter is typically 5 nucleotides to 4000 nucleotides in length or longer, e.g., 10 nucleotides to 2000 nucleotides in length or longer, 20 nucleotides to 1000 nucleotides in length or longer, 30 nucleotides to 500 nucleotides in length or longer, 40 nucleotides to 300 nucleotides in length or longer, e.g., 50 nucleotides to 200 nucleotides in length or longer, 60 nucleotides to 100 nucleotides in length or longer, or 70 nucleotides to 90 nucleotides in length or longer. In some embodiments, a promoter featured in the invention is less than 5 nucleotides in length. In some embodiments, a promoter can be 5 nucleotides to 50 nucleotides in length, 10 nucleotides to 60 nucleotides in length, 20 nucleotides to 70 nucleotides in length, 50 nucleotides to 150 nucleotides in length, 60 nucleotides to 200 nucleotides length or 80 nucleotides to 250 nucleotides in length, 100 nucleotides to 500 nucleotides in length or 300 nucleotides to 1000 nucleotides in length. The test promoters featured in the invention are typically, e.g., 5 nucleotides, 20 nucleotides, 50 nucleotides, 100 nucleotides, 150 nucleotides, 200 nucleotides, 300 nucleotides, 500 nucleotides, 1000 nucleotides, 2000 nucleotides, 3000 nucleotides in length or longer.

As promoters are typically immediately adjacent to the gene in question, positions in the promoter are designated relative to the transcriptional start site, where transcription of RNA begins for a particular gene, such as a gene encoding a reporter protein. Thus, positions upstream of the gene are negative numbers counting back from −1, for example −100 is a position 100 base pairs upstream of the gene whose expression is regulated. A promoter featured in the present invention is typically located at least 35 nucleotides upstream of the transcription start site of the gene, e.g., the gene encoding a fluorescent protein. The test promoter can begin, e.g., −40, −50, −70, −80 or −100 nucleotides or more upstream from the transcriptional start site.

A test promoter for use in the methods described herein can include adjacent enhancer or repressor elements, such as elements that function with the test promoter, e.g., in a circuit or in a biological system. Enhancer elements can be bound by protein or RNA (e.g., microRNA (miRNA)), to influence the strength or activity of a promoter. For example, in some embodiments, a protein(s) or miRNA can bind an enhancer element near the promoter to activate or repress the promoter. Such enhancer elements can be included in the “test element” construct as described herein. Promoters, e.g., test promoters, that can be evaluated by the methods featured in the invention include, e.g., inducible, activatable, repressible and hybrid promoters. As used herein, a “hybrid promoter” can contain multiple elements, e.g., multiple synthetic elements, or elements from different promoter regions. A hybrid promoter can contain one or more enhancer elements and one or more activation sequences, and/or elements that are differentially regulated.

A test promoter can be designed and synthesized de novo, or Obtained from a commercially or publicly available collection, such as from the Registry of Standard Biological Parts (on the web at partsregistry.org) at the BioBricks Foundation collection (on the web at biobricks.org). In one embodiment, a test promoter is obtained from the biofab collection (on the web at biofab.org). A control promoter can also be designed or synthesized de novo, or obtained from a commercially or publically available collection, such as the BioBricks collection or the biofab collection.

A “control promoter” can be any promoter that has a predictable and detectable expression level in a given cell type and under particular cell culture conditions. These predictable and detectable expression levels provide a basis for comparison for a promoter whose strength is unknown, e.g., in the context of the particular circuit being tested, which for example, is dependent on the other DNA parts in the circuit. A control promoter can be a known, naturally occurring promoter, e.g., a constitutive promoter, such as a constitutive viral promoter. The control promoter can also be any identified DNA sequence (naturally occurring or engineered) that is found to provide a predictable and detectable expression level in a given cell type and under particular cell culture conditions. A naturally occurring promoter can be from any organism, e.g., a viral, bacterial, plant, or mammalian organism.

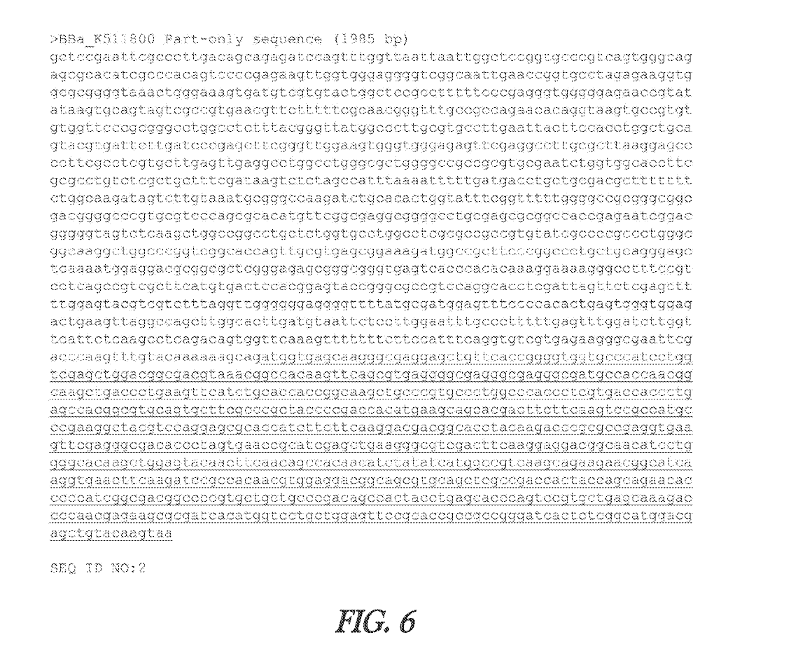

A control promoter is typically a constitutive promoter. “Constitutive” promoters are those that drive expression continuously under most environmental conditions and states of development or cell differentiation. An exemplary constitutive promoter that is useful as a control promoter is the Hef1a promoter. For example, the DNA part available as BioBricks reference BBa_K511801 includes the Hef1a promoter fused to the mKATE fluorescent protein gene (FIG. 5). The DNA part available BioBricks reference BBa_K511800 includes the Hef1a promoter fused to the ERFP2 fluorescent protein gene (FIG. 6). The Hef1a promoter sequence is available as a DNA part at, e.g., BioBricks reference BBa_K415508 (FIG. 10).

As used herein, the term “relation” refers to a comparison between two sets of values. Examples of relations between two values include quantitative measures such as ratio, difference, or absolute magnitude and qualitative measures such as greater than, less than, equal to, and their negations. Values can be described in terms of particular sampling times or the evolution of values over time.

As used herein, a “reporter” gene or protein is any gene or protein that is easily detectable and measurable. Exemplary reporter proteins include, e.g., fluorescent proteins, chemiluminescent proteins, proteins that can be detected by immunostaining, and radioactively-labeled proteins.

Exemplary fluorescent proteins include, e.g., enhanced blue fluorescent protein (EBFP), enhanced blue fluorescent protein-2 (EBFP2), mKATE, iRFP (infrared fluorescent protein), enhanced yellow fluorescent protein (EYFP), yellow fluorescent protein (YFP), Katustika, Ds-Red express, TurboRFP, TagRFP, green fluorescent protein (GFP), blue fluorescent protein (BFB), cyan fluorescent protein (CFP), enhanced green fluorescent protein (EGFP), AcGFP, TurboGFP, Emerald, Azami Green, ZsGreen, Sapphire, T-Sapphire, enhanced cyan fluorescent protein (ECFP), mCFP, Cerulean, CyPet, AmCyan1, Midori-Ishi Cyan, mTFP1 (Teal), Topaz, Venus, mCitrine, YPet, PhiYFP, ZsYellow1, mBanana, Kusabira Orange, mOrange, dTomato, dTomato-Tandem, DsRed, DsRed2, DsRed-Express (T1), DsRed-Monomer, mTangerine, mStrawberry, AsRed2, mRFP1, JRed, mCherry, HcRed1, mRaspberry, HcRed1, HcRed-Tandem, mPlum, and AQ143.

Fluorescent proteins can be assayed, e.g., by FACS or fluorescence microscopy.

Other exemplary reporter proteins include beta-galactosidase (encoded by the lacZ gene), any polypeptide comprising a detectable protein tag, such as a FLAG tag or HISx6 tag, a c-myc tag or a HaloTag® (Promega Corporation).

Reporter gene expression can be assayed by immunohistochemistry, e.g., by detecting expressed proteins with antibodies labeled with different detectable probes (e.g., Alexa Fluor®, Oregon Green® or Pacific Blue®; horseradish peroxidase (HRP) and alkaline phosphatase (AP)). In one embodiment, beta-galactosidase is assayed using X-Gal substrate.

In one embodiment, a test reporter protein and a control reporter protein can be labeled with different radioisotopes, e.g., ³²P, ¹²⁵I, or ³⁵S, such as by culturing cells in the presence of the isotopes. The differential labeling of the different isotopes on the control and test reporter proteins can be assayed, e.g., by mass spectrometry.

A “reporter protein transcription unit” as used herein, is a protein coding sequence of DNA or RNA in the absence of native upstream (and optionally, downstream) regulatory sequences. In the present invention, the transcription units of reporter genes are placed under control of selected test and control promoters, and therefore native sequences that influence gene expression under native conditions are removed so as not to interfere with the controlled expression system. The terms “transcription unit” and “gene” are used interchangeably herein.

The test genetic element and the control genetic element can be on the same or different plasmids. When a test genetic element and a control genetic element are on the same nucleic acid molecule, e.g., plasmid, the test genetic element/test reporter protein transcription unit and the control genetic element/control reporter protein transcription unit can be in either order on the plasmid. For example, a test promoter/test fluorescent protein transcription unit can be upstream or downstream of a control promoter/control fluorescent protein transcription unit. An insulator sequence is optionally included between the test genetic element/test reporter protein transcription unit and the control genetic element/control protein transcription unit. An “insulator” sequence blocks any affects of the test or control element on each other, such that the test element only affects transcription of its designated reporter gene (e.g., the “test” reporter gene), and the control element only affects transcription of its designated reporter reporter gene (e.g., the “control” reporter gene). The nucleic acid comprising a test genetic element/test reporter protein transcription unit, and a control genetic element/control reporter protein transcription unit are typically not integrated into the genome of the cell.

Suitable cells for use in assaying test genetic elements include eukaryotic cells (e.g., yeast, fungi, insect, plant, mammalian, or nucleated cells from other multicellular organisms) and prokaryotic cells (e.g., E. coli (Escherichia coil), Bacillus subtilis). Exemplary mammalian cells include Hek293, CHO (Chinese Hamster Ovary Cells), 3T3, HeLa, COS-7, Balb/c and the like.

Suitable plasmids for evaluating a test element/test reporter protein transcription unit can include a selection gene, also called a selectable marker. Typical selection genes encode proteins that (a) confer resistance to antibiotics or other toxins, e.g., ampicillin, neomycin, streptomycin, methotrexate, or tetracycline; (b) complement auxotrophic deficiencies; or (c) supply critical nutrients not available from complex media, e.g., the gene encoding D-alanine racemase for Bacilli.

Optionally, suitable selectable markers for mammalian cells are those that enable the identification of cells competent to take up the plasmid(s) comprising the test and control promoters, such as Dihydrofolate reductase (DHFR), neo, glutamic pyruvate transaminase (gpt), hygromycin (hygro), thymidine kinase (tk), hypoxanthine-guanine phosphoribosyltransferase (hgprt), or adenine phosphoribosyltransferase (aprt). An appropriate host cell when wild-type DHFR is employed is the CHO cell line deficient in DHFR activity, prepared and propagated as described by Urlaub et al., Proc. Natl. Acad. Sci. USA 77:4216 (1980). A suitable selection gene for use in yeast is the trp1 gene present in the yeast plasmid YRp7 (Stinchcomb et al., Nature 282:39 (1979); Kingsman et al., Gene 7:141 (1979); Tschemper et al., Gene 10:157 (1980)). The trp1 gene provides a selection marker for a mutant strain of yeast lacking the ability to grow in tryptophan, for example, ATCC No. 44076 or PEP4-1 (Jones, Genetics 85:12 (1977)).

There are a variety of techniques available for introducing nucleic acids into viable cells, e.g., for introducing a plasmid comprising a test promoter/test reporter protein transcription unit and a control promoter/control reporter protein transcription unit. Techniques suitable for the transfer of nucleic acid into mammalian cells in vitro include the use of liposomes, electroporation, microinjection, cell fusion, DEAE-dextran, calcium phosphate precipitation method, etc. A variety of known methods, including electroporation or calcium chloride methods can be used to transfer nucleic acids into prokaryotic cells, e.g., E. coli.

In one embodiment, a virus is used to deliver nucleic acids into a cell.

In one embodiment, more than one genetic element is assayed at the same time, e.g., by comparing expression levels of the test reporter and control reporter expression levels. The test genetic elements can be on the same plasmid or different plasmids, and at least one of the test genetic elements can be on the same plasmid as the control plasmid, or each of the test genetic elements and the control element can be on separate plasmids.

In one embodiment, more than one test element and control element pair are assayed at the same time, e.g., in the same cell culture. In this embodiment, a different reporter gene is used for each test and control element. For example, a first test element/control element pair can include two different fluorescent reporter genes (e.g., EBFP2 and GFP), and a second test element/control element pair can include two different fluorescent reporter genes that also differ from the fluorescent reporter genes coupled with the first test element/control element pair. In another embodiment, the one control element is used with a first test element and a second test element, all three elements using different fluorescent reporter genes.

In one embodiment the plasmid containing the test element/test reporter protein transcription unit, and the control element/control reporter protein transcription unit comprises an origin of replication, e.g., for replication in a bacterial cell.

In one embodiment the plasmid containing the test genetic element/test reporter protein transcription unit, and the control genetic element/control reporter protein transcription unit, does not replicate inside a cell, e.g., a mammalian cell. Thus, after the plasmid is transfected into cells, e.g., mammalian cells, the number of plasmids per cell is halved, until eventually, there is no more than one plasmid per cell. When the test genetic element/test reporter protein transcription unit, and the control genetic element/control reporter protein transcription unit are cloned onto separate plasmids, cells that contain one plasmid comprising the test element/test reporter protein transcription unit, and one plasmid comprising the control element/control reporter protein transcription unit are evaluated. Cells comprising one of each plasmid are identified by growth on selective media and by FACS.

In one embodiment the plasmid containing the test genetic element/test reporter protein transcription unit, and the control genetic element/control reporter protein transcription unit, does not replicate inside a cell, e.g., a mammalian cell. However, the plasmid includes a selectable marker, and the nucleic acid comprising the marker and the test genetic element/test reporter protein transcription unit, and the control genetic element/control reporter protein transcription unit, integrates into the genome of the cell.

A plasmid comprising a test genetic element/test reporter protein transcription unit, and a control genetic element/control reporter protein transcription unit can be transfected into a cell to evaluate the test element, e.g., to evaluate the expression of the test element. When the test element/test reporter protein transcription unit, and the control element/control reporter protein transcription unit are cloned onto separate plasmids, the plasmids are cotransfected into cells. The cells can be eukaryotic cells, e.g., mammalian cells, e.g., Hek293 cells or Chinese Hamster Ovary (CHO) cells. In certain embodiments, the plasmids are transformed into prokaryotic cells, e.g., E. coli cells.

Expression of the test genetic element, e.g., test promoter, can be performed in a variety of cell types or in a variety of cell culture conditions, but the evaluation of the test element is always relative to the activity of the control genetic element, which is expressed in the same cell, and accordingly, in the same culture conditions.

In some embodiments, the characterization process is based on FACS, which allows large numbers of single cell fluorescence measurements to be obtained quickly. These single cell measurements capture variation in device behavior, which is necessary when composing devices within a cell. In this process the control reporter enables the measurement of the number of copies of a system in the cell (often introduced by the high variability in number of transfected or transformed plasmids).

In some embodiments, individual plasmid behavior is measured using a cotransfection or cotransformation of several species of plasmid. This can increase the speed and reliability of characterization by eliminating the many slow wet-lab steps previously required by the need for a single plasmid circuit and/or single copy of a plasmid.

Libraries

The invention provides methods for screening for an optimal promoter by providing a library of candidate genetic elements, e.g., candidate DNA promoters. As used herein, a “library” is a collection of entities that includes nucleic acid fragments that contain potential elements, e.g., potential DNA promoter sequences. Each fragment contains nucleotides that are varied among the collection of entities, such that different nucleotides are represented at various positions of the fragment. Each candidate genetic element, e.g., candidate DNA promoter, in the library can be stored on an individual plasmid. The plasmids can include at least one selectable marker, e.g., for selection in bacteria, and optionally for selection in mammalian cells. The plasmids can also have a prokaryotic origin of replication, such as for propagation in bacterial cells.

In one embodiment, methods of making a library are provided. Methods of making the library include providing a plurality of test genetic elements, and introducing each test genetic element of the plurality into a plasmid, where each test genetic element of the plurality is positioned upstream of (i.e., 5′ to) a first reporter gene, where each first reporter gene is positioned 5′ to a control genetic element that is positioned 5′ to a second control reporter gene. Methods of making the library also include providing a plurality of test genetic elements, and introducing each test genetic element of the plurality into a plasmid, where each test genetic element of the plurality is positioned downstream of (i.e., 3′ to) a first reporter gene, and where optionally each first reporter gene is positioned 3′ to a control genetic element that is positioned 3′ to a second control reporter gene. The selection of 3′ or 5′ depends on the genetic element to be tested: for example, miRNA, ribozymes, and degradation tags will typically use 3′ and promoters and RBS will typically use 5′, though details may vary with the particular element and intended purpose of the test. The test genetic element/first report gene can be separated from the control element/second reporter gene by a spacer sequence, and the spacer sequence can optionally include an insulater sequence. Each test genetic element can be introduced into the plasmid by a restriction enzyme based method or by a recombination cloning method.

The plurality of test genetic elements can be obtained from a commercial source or a publicly available source, or can be designed and synthesized de novo, such as by a directed design of elements. In one embodiment, random mutagenesis e.g., by a chemical or enzymatic method, such as PCR mutagenesis) is used to generate a plurality of test elements, e.g., test promoters, for screening in a library.

In one embodiment, the first reporter gene and the second reporter gene encodes a first and second fluorescent protein, respectively.

Screening methods can include, for example, transforming the plurality of test genetic elements, which are coupled to a control element/control reporter gene, or a subset of the plurality, into cells, e.g., mammalian cells, and selecting an element that causes cells to express the first reporter gene such that the relation of test reporter gene expression to control reporter gene expression exceeds a designated value (e.g., if a stronger element is desired), or is below a designated value (e.g., if a weaker element is desired). For example, a test promoter that causes expression of a test fluorescent protein such that the relation of fluorescence of the test protein to fluorescence of the control protein exceeds a designated amount, then the test promoter can be selected for use in a circuit.

In one embodiment, a plasmid in a library that contains a test promoter has a multiple cloning site (MCS), or specific restriction sites, downstream of the test genetic element, e.g., for cloning of a desired reporter gene. The test element/reporter gene (i.e., test element/test fluorescent protein gene), can then be removed as one DNA fragment and cloned into a second plasmid that contains a control element that drives expression of a second reporter gene (i.e., control element/control reporter transcription unit). The plasmid containing both the test element/test reporter gene and the control element/control reporter gene can then be transformed into a bacterial cell for propagation or for evaluation of the test promoter, or can be transfected into a eukaryotic cell, e.g., a mammalian cell, for evaluation of the test element.

In one embodiment, a plasmid in a library that contains a test promoter has a multiple cloning site (MCS), or specific restriction sites, downstream of the test genetic element, and a second plasmid contains a first reporter gene, a spacer region, and a control element that drives expression of a second reporter gene (i.e., control element/control reporter transcription unit). The first reporter gene, spacer region, and control element/control reporter transcription unit can be removed as one DNA fragment and cloned into the plasmid that contains the test element, such that the test element (e.g., test promoter) drives expression of the first reporter gene. The resulting plasmid containing both the test element/test reporter protein transcription unit and the control element/control reporter transcription unit can then be transformed into a bacterial cell for propagation or for evaluation of the test promoter, or can be transfected into a eukaryotic cell, e.g., a mammalian cell, for evaluation of the test element.

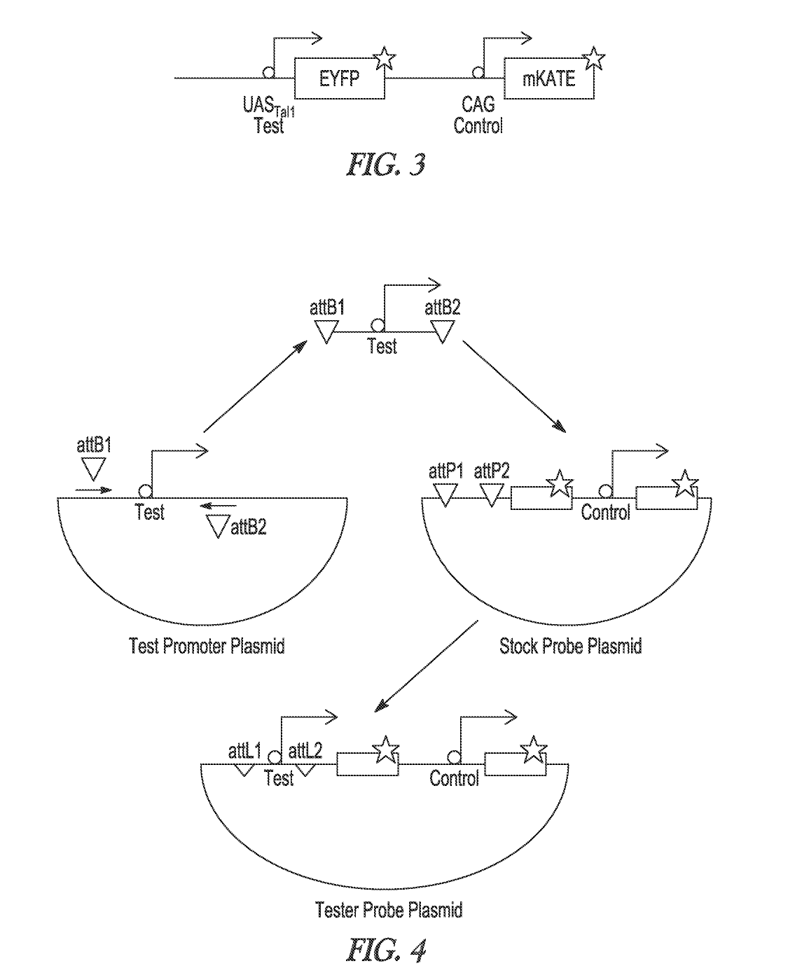

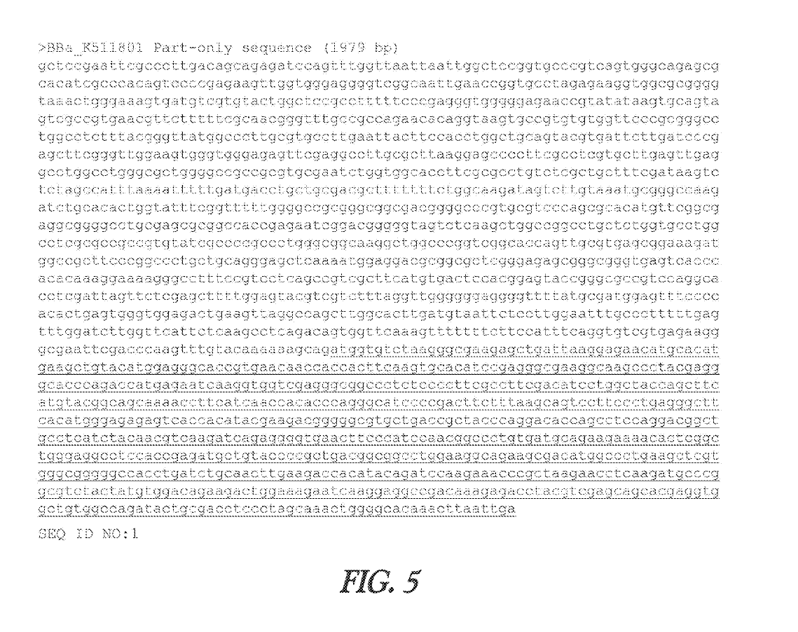

In yet another embodiment, a plasmid in a library that contains a first reporter gene, a spacer region, and a control element that drives expression of a second reporter gene (i.e., control element/control reporter transcription unit) has a multiple cloning site, or specific restriction enzyme sites, upstream of (i.e., 5′ to) the first reporter gene. This plasmid is an example of a “stock probe plasmid” (see FIG. 4). The test genetic element can then be cloned into the plasmid that contains the first reporter gene, spacer region, and a control element that drives expression of a second reporter gene, such that the test element (e.g., test promoter) drives expression of the first reporter gene. The resulting plasmid containing both the test element/test reporter protein transcription unit and the control element/control reporter transcription unit can then be transformed into a bacterial cell for propagation or for evaluation of the test promoter, or can be transfected into a eukaryotic cell, e.g., a mammalian cell, for evaluation of the test element.

In one embodiment, a commercially available system, such as the BioBrick™ Assembly Kit, by New England BioLabs® Inc., provides plasmids useful for cloning genetic elements by restriction enzyme-based methods for use in a library construction and screening methods as described herein.

In one embodiment, recombination cloning, instead of restriction enzyme-based cloning, is used to generate a vector containing a test element/test fluorescent transcription unit and control element/control reporter transcription unit. Recombination cloning involves the use of recombination enzymes, e.g., bacteriophage recombination enzymes, that facilitate the insertion of a sequence of interest between two specific recombination sites (see, for example, the Gateway® Cloning system by Invitrogen®).

In an exemplary recombination cloning method, the enzymes involved in the lysogenic cycle of the lambda bacteriophage (integrase (Int), excisionase (Xis) and integration host factor (IHF)) are utilized. Integrase, catalyzed by IHF, can mediate excision of sequences out of an E. coli genome and integration of sequences into an E. coli genome by using recombination sequences such as attB, attP, attL and attR. In a host E. coli cell, the integrase catalyzes the recombination of attB and attP sites to form attL and attR sites in the genome at the site of integration. The opposite reaction takes place during excision: Int catalyzes the recombination of attL and attR sites to generate an attP site on the excised fragment, and an attB site in the host genome. The reaction equilibrium normally favors attB and attP recombination, and the excisionase Xis catalyzes excision by binding to the attR site and shifting equilibrium to favor the reverse reaction.

This technology can be used to facilitate cloning of, e.g., a test element, such as a test promoter, into a control vector as described above, e.g., comprising a test reporter transcription unit, a spacer sequence, and a control promoter/control reporter transcription unit. In one embodiment, the cloning reaction requires combining equimolar parts of a test element comprising flanking attB sequences at each end of the element (e.g., a PCR fragment comprising the test element, where the PCR fragment is generated using primers that incorporate the attB sequences at the ends of the fragment) and a vector comprising the test reporter transcription unit, a spacer sequence, and a control promoter/control reporter transcription unit (where the sequence comprising the test reporter transcription unit, a spacer sequence, and a control promoter/control reporter transcription unit is flanked by attP sites), with a mix of integrase, IHF and excisionase, to catalyse the recombination reaction. The resulting plasmid, e.g., a “tester probe plasmid,” can be directly transfected into a cell, e.g. a mammalian cell, for evaluation of the test element (see, e.g., FIG. 4).

The tester probe plasmid can be a plasmid that is suitable for transfection into a eukaryotic cell (e.g., a mammalian cell) for evaluation of the test genetic element, e.g., a test promoter. For example, the plasmids of the library (e.g., “test promoter plasmids”) can include a selectable marker gene for use in eukaryotic cells.

The DNA fragment containing a first fluorescent protein gene, optionally an insulator sequence, and a control genetic element, e.g., a control promoter that regulates expression of a second reporter protein gene can be obtained from a “stock probe plasmid” that is widely useful for the methods described herein. The present invention provides a stock probe plasmid comprising a first reporter protein gene, optionally an insulator sequence, and a control element that regulates expression of a second reporter protein gene that can be cloned into the MCS of any variety test element plasmids, such as those obtained from any source, such as from any library, or collection or de novo construction.

Methods of Measuring Reporter Gene Expression

As provided by the invention, the strength of a test genetic element, e.g., a test promoter, can be measured by comparing the strength of expression of a first reporter protein, which is controlled by the test element, with the strength of expression of a second reporter protein, which is under control of a control genetic element.

In one embodiment, expression of the first and second fluorescent proteins can be assayed by any method known in the art. For example, fluorescence can be measured by fluorescence activated cell sorting (FACS), by microscopy, fluorometry, immunohistochemistry, or mass spectrometry. Fluorescence can also be measured by a combination of these and other methods. Microscopy, thr example, provides single-cell measurements with optimal time resolution. The ability to measure single cell fluorescence by microscopy is also enhanced when coupled with machine vision algorithms. Typically, fluorescence is measured at least by a method that includes FACS. The use of FACS allows large numbers of single-cell fluorescence measurements to be obtained quickly.

A fluorescent protein under control of test genetic element, e.g., a test promoter, can be any fluorescent protein, such as enhanced blue fluorescent protein (EBFP), enhanced blue fluorescent protein-2 (EBFP2), mKATE, iRFP (infrared fluorescent protein), enhanced yellow fluorescent protein (EYFP), fluorescent protein (YFP), Katushka, Ds-Red express, TurboRFP, TagRFP, green fluorescent protein (GFP), blue fluorescent protein (BFP), cyan fluorescent protein (CFP), enhanced green fluorescent protein (EGFP), AcGFP, TurboGFP, Emerald, Azami Green, ZsGreen, Sapphire, T-Sapphire, enhanced cyan fluorescent protein (ECFP), mCFP, Cerulean, CyPet, AmCyan1, Midori-Ishi Cyan, mTFP1 (Teal), Topaz, Venus, mCitrine, YPet, PhiYFP, ZsYellow1, mBanana, Kusabira Orange, mOrange, dTomato, dTomato-Tandem, DsRed, DsRed2, DsRed-Express (T1), DsRed-Monomer, mTangerine, mStrawberry, AsRed2, mRFP1, JRed, mCherry, HcRed1, mRaspberry, HcRed1, HcRed-Tandem, mPlum, and AQ143.

A fluorescent protein under control of control genetic element, e.g., a control promoter, can be any fluorescent protein, such as enhanced blue fluorescent protein (EBFP), enhanced blue fluorescent protein-2 (EBFP2), mKATE, iRFP (infrared fluorescent protein), enhanced yellow fluorescent protein (EYTP), yellow fluorescent protein (YFP), Katushka, Ds-Red express, TurboRFP, TagRFP, green fluorescent protein (GFP), blue fluorescent protein (BFP), cyan fluorescent protein (CFP), enhanced green fluorescent protein (EGFP), AcGFP, TurboGFP, Emerald, Azami Green, ZsGreen, Sapphire, T-Sapphire, enhanced cyan fluorescent protein (ECFP), mCFP, Cerulean, CyPet, Midori-Ishi Cyan, mTFP1 (Teal), Topaz, Venus, mCitrine, YPet, PhiYFP, ZsYellow1, mBanana, Kusabira. Orange, mOrange, dTomato, dTomato-Tandem, DsRed, DsRed2, DsRed-Express (T1), DsRed-Monomer, mTangerine, mStrawberry, AsRed2, mRFP1, JRed, mCherry, HcRed1, mRaspberry, HcRed1, HcRed-Tandem, mPlum, and AQ143.

The reporter protein under control of the test genetic element is different than the reporter protein under control of the control genetic element.

In one embodiment, the reporter gene under control of the test genetic element and the reporter gene under control of the control genetic element are fluorescent proteins, and the fluorescent proteins thr each element are chosen so as to have minimal spectral overlap. For example, the fluorescent proteins EBFP2, mKATE, iRFP and EYFP have minimal spectral overlap with one another, and therefore any one fluorescent protein chosen for use with a test element can be coupled for use with any of the other three fluorescent proteins for use with a control element. In one embodiment, two of the set of fluorescent proteins comprising EBFP2, mKATE, iRFP and EYFP are used in mammalian cells to test a genetic element in the coupled expression system featured in the invention.

In another example, the fluorescent proteins GFP, RFP and BFP have minimal spectral overlap with one another, and therefore any one fluorescent protein chosen for use with a test element can be coupled for use with any of the other two fluorescent proteins for use with a control element. In one embodiment, two of the set of fluorescent proteins comprising GFP, RFP and RFP are used in prokaryotic cells (e.g., E. coli) to test a genetic element in the coupled expression system featured in the invention.

The use of FACS for the evaluation of a test element, e.g., a test promoter, requires a normalized FACS characterization. Any method for normalization of FACS data can be used for the analysis. For example, by one method of FACS analysis, fluorescence color correction is required, at least because a few percent of the measured level of one fluorescent protein (e.g., the control protein) will often be added to the measured level of another fluorescent protein (e.g., the test protein). The interference caused by the overlap in fluorescence signal can be corrected by a linear subtraction method.

Kits

Plasmids and reagents required to practice the claimed methods can be provided in a kit. In one embodiment, the kit includes (a) a container that contains a plasmid(s) comprising a control genetic element/control reporter protein transcription unit, and optionally, a test reporter protein transcription unit; optionally (b) reagents and buffers for introducing the plasmid(s) into a cell, optionally (c) cells (e.g., mammalian or bacterial cells) for use with the plasmid(s), and optionally (d) informational material.

The informational material can be descriptive, instructional, marketing or other material that relates to the methods described herein and/or the use of the agents for evaluating a DNA part, e.g., a DNA promoter. In one embodiment, the informational material includes information about one or more of cloning a test genetic element, e.g., a test promoter, into the plasmid comprising the control and test reporter probes, or cloning the test and reporter probes into a plasmid comprising a test genetic element, to create a tester probe plasmid; transforming a tester probe plasmid into a cell type, e.g., one or more of a bacterial or mammalian cell type; evaluating the fluorescence signals produced from the tester probe plasmid and analyzing the results, e.g., to arrive at a conclusion regarding the strength of the test genetic element; concentration; date of expiration; batch or production site information; and so forth.

The informational material of the kits is not limited in its form. The information can be provided in a variety of formats, including printed text, computer readable material, video recording, or audio recording, or information that provides a link or address to substantive material. The informational material can be provided on a label, e.g., on a vial comprising a plasmid.

In addition to a plasmid, the composition in the kit can include other ingredients, such as a solvent or buffer, a stabilizer, or a preservative. The components of the kit (e.g., plasmid(s)) can be provided in any form, e.g., a liquid, dried or lyophilized form, and substantially pure and/or sterile. When the agents are provided in a liquid solution, the liquid solution is typically an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing a reagent, e.g., a plasmid. In some embodiments, the kit contains separate containers, dividers or compartments for the composition and informational material. For example, a composition comprising a plasmid, or a cell, can be contained in a bottle or vial or tube, and the informational material can be contained in a plastic sleeve or packet. In other embodiments, the separate elements of the kit are contained within a single, undivided container. For example, the composition is contained in a bottle or vial or tube that has attached thereto the informational material in the form of a label. In some embodiments, the kit includes a plurality (e.g., a pack) of individual containers, each containing one or more unit dosage forms (e.g., a dosage form described herein) of the agents. The containers of the kits can be air tight, waterproof (e.g., impermeable to changes in moisture or evaporation), and/or light-tight.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Evaluation of a Test DNA Promoter by Comparison to Activity of a Control Promoter

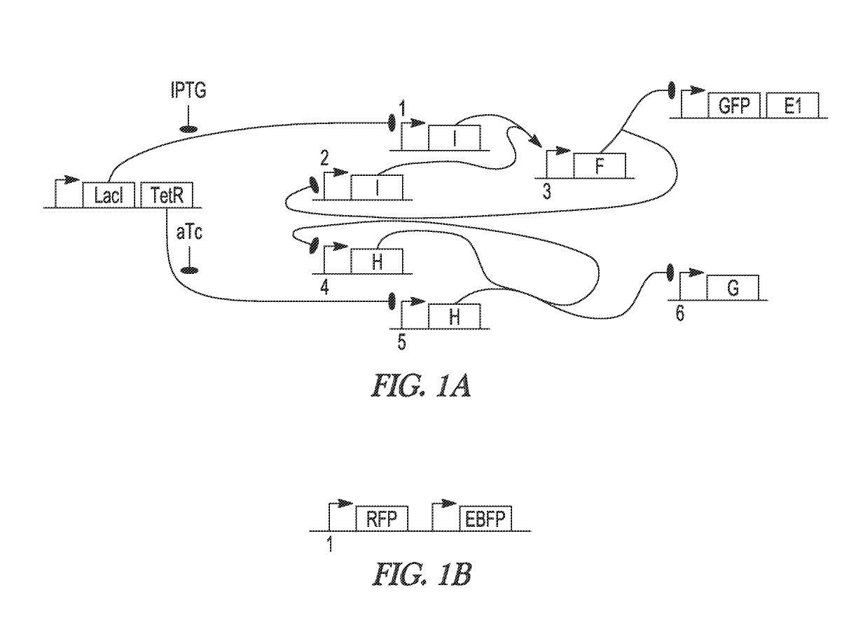

A test DNA promoter can be coupled to a control DNA promoter to evaluate the test promoter (e.g., the strength or activity of the test promoter) for use in a circuit. FIG. 1A illustrates a typical circuit, which causes the expression of GFP and E1 or G protein, depending on whether IPTG or aTc is present. The promoters are indicated by rightward pointing arrows and are identified by numbers.

FIG. 1B is a diagram illustrating a method of evaluating promoter 1, by placing the fluorescent protein RFP under control of the promoter 1, and operably linking the test promoter/RFP transcription unit to a control promoter/second fluorescent reporter protein EBFP.

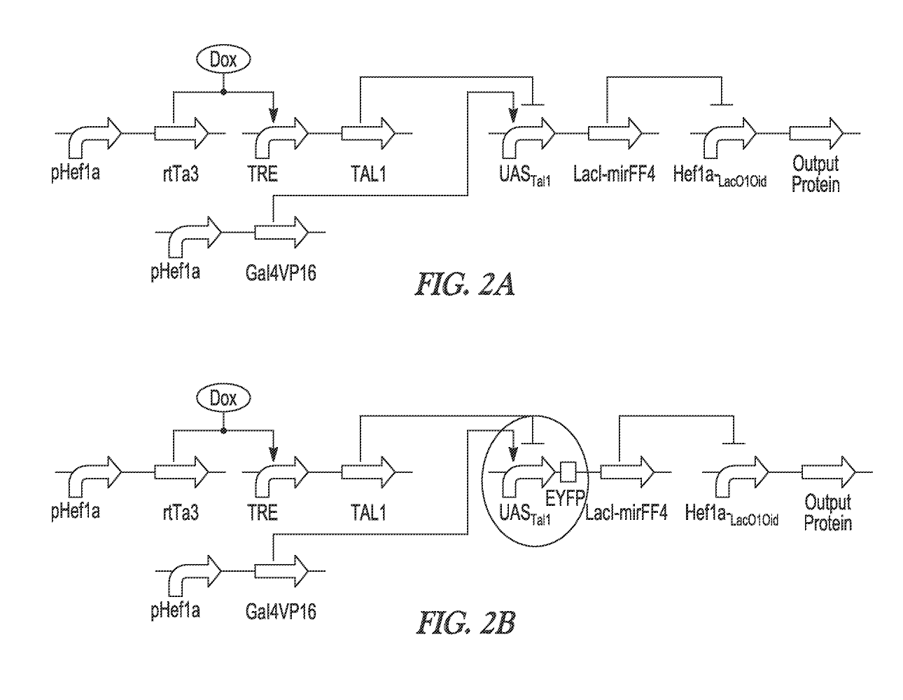

FIG. 2A is a diagram of a circuit that includes a regulatory sequence (UAS_{TAL1}) that is differentially regulated by Tal1 protein and Gal4VP16: TAL1 protein binds the UAS element causing decreased activity of the element and a subsequent inhibition of expression of LacI-mirFF4 gene. GAL4VP16, however, activates the UAS element, causing upregulation of LacI-mirFF4 expression.

FIG. 2B illustrates a prior approach for evaluating activity of the UAS_{TAL1} regulatory element. By the prior method, the circuit was debugged reassembling the circuit with the insertion of a fluorescent protein (EYFP) after the UAS_{TAL1} element. This process would require days to weeks to complete.

By the new method described here, the UAS_{TAL1} element is evaluated by cloning the element upstream (5′ to) a reporter gene, such as the EYFP gene shown in FIG. 3. The UAS_{TAL1}/EYFP construct is expressed with a linked control element (CAG) constitutive promoter, which regulates expression of a second fluorescent protein, mKate, (FIG. 3).

### Example 2

### Test Promoters and Control Promoters, and Fluorescent Reporter Constructs are Available from the BioBricks Foundation Collection (on the Web at biobricks.org

The following promoters are exemplary:

Control construct: Blue Fluorescent Protein Generator (Hef1a-EBFP2) (BioBricks reference BBA_k511 800; FIG. 6); 1985 basepairs long.

Control construct: Red Fluorescent Protein Generator (Hef1a-mKATE) (BioBricks reference BBA_k511 801; FIG. 5); 1979 basepairs long.

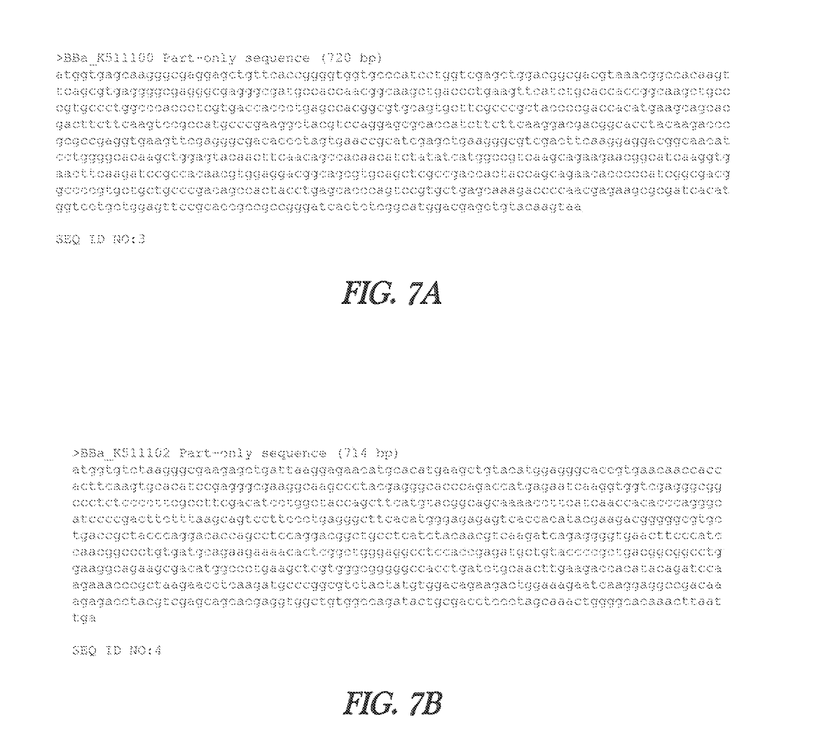

Blue fluorescent protein reporter: Enhanced Blue Fluorescent Protein 2 (EBFP2) (BioBricks reference BBA_k511100; FIG. 7A); 720 basepairs long.

Red fluorescent protein reporter: mKATE (BioBricks reference BBA_k511102; FIG. 7B); 714 basepairs long.

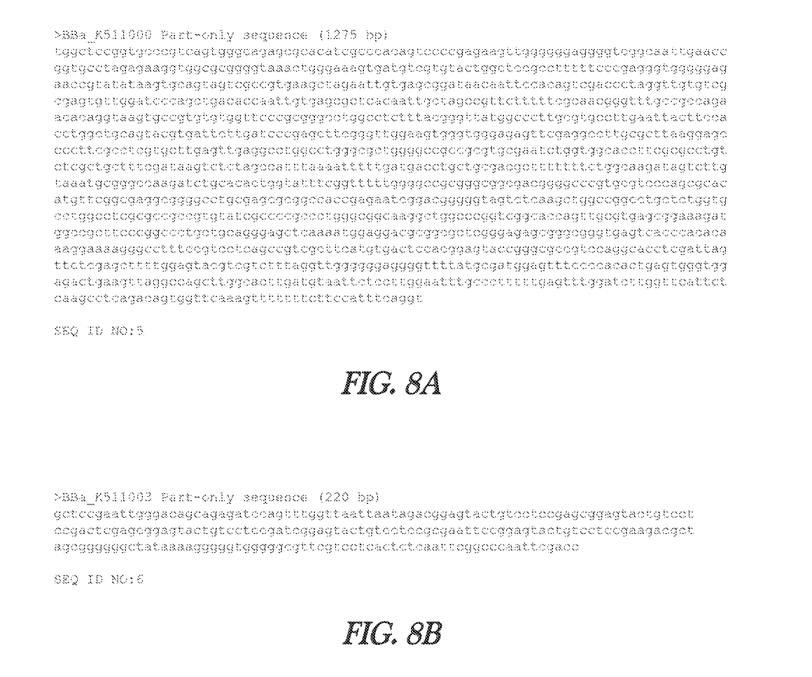

Test promoter: Hef1A-LacOid (BioBricks reference BBA_k511000; FIG. 8A) 1275 basepairs long; encodes a promoter with low-level, constitutive activity that can be repressed by variants of the LacI transcriptional repressor. This part has been modified to add repressibility to the existing well-known constitutive promoter. In addition, different domains can be added onto the LacI repressor or IPTG can be added, allowing the user to toggle and tune the amount of repression, significantly increasing the flexibility and usage of the existing part. Cotransfection of a construct containing a yellow fluorescent protein (EYFP) driven by the Hef1a-LacO promoter with a construct containing a LacI-KRAB transcriptional repressor driven by the TRE-Tight promoter proves that it is possible to repress Hef1a-LacO expression with the LacI-KRAB LacI variant, which includes a Kruppel-associated box motif previously shown to silence chromatin and thereby inhibit transcription.

Test promoter: UAS-GAL4 (BioBricks reference BBA_k511003; FIG. 8B) 220 basepairs long; inducible by variants of the Gal4 transactivator and off otherwise.

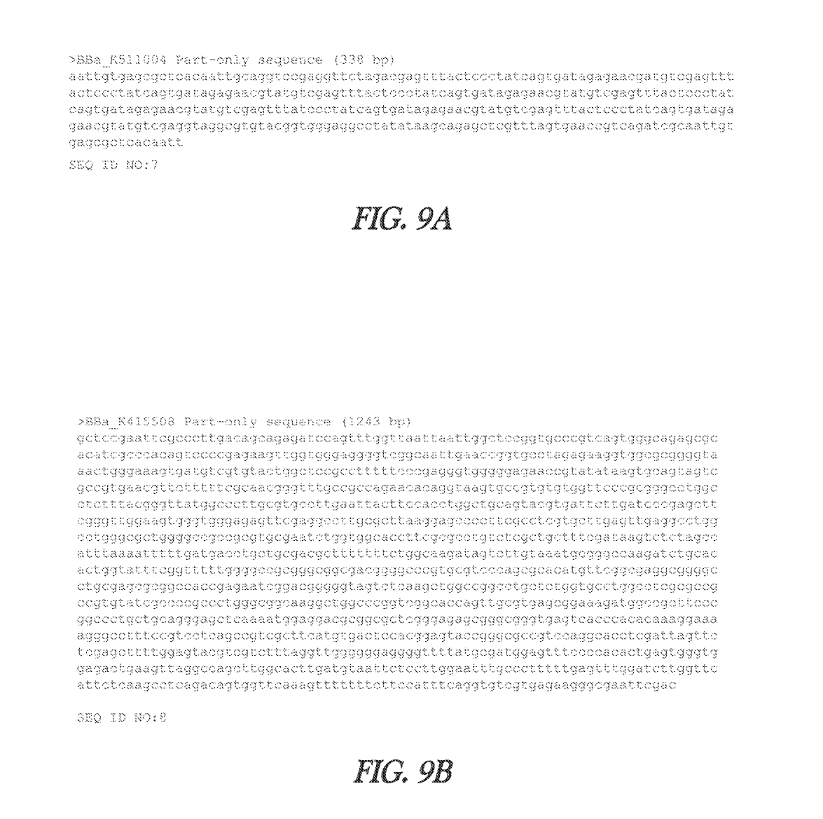

Test promoter: TRE-Tight-LacOid promoter (BioBricks reference BBA_k51.1004; FIG. 9); 338 basepairs long; encodes an inducible promoter that can be activated by tTA/rtTA transactivator variants in the presence of tetracycline analogues and can be repressed by variants of the LacI transcriptional repressor.

Parts described above are assembled into a vector using recombination cloning (Deming et al., “BBF RFC #65: Recombination Based Part Assembly” Mammoblock RFC, Oct. 24, 2010). For example, a Hef1A-LacOid (BioBricks reference BBA_k511000) test promoter is cloned upstream of the Blue fluorescent protein reporter EBFP2 (BioBricks reference BBA_k511100), and this construct is cloned upstream of the constitutive promoter construct Hef1a-mKATE (BioBricks reference BBA_k511 801).

To create the expression construct, the Gateway® cloning system from Invitrogen™ is used according to the manufacturer's protocol.

The resulting plasmid is then transfected into mammalian cells and the relation of fluorescence of the test and the control promoters is measured to determine the strength of the test promoter.

In other exemplary constructions:

(i) a Hef1A-LacOid (BioBricks reference BBA_k5111000; FIG. 8A) test promoter is cloned upstream of the Red Fluorescent Protein Generator (mKATE) (BioBricks reference BBA_k511102; FIG. 7B), and this construct is cloned upstream of the reporter construct Hef1a-ERFP2 (BioBricks reference BBA_k511800; FIG. 6).

(ii) a UAS-GAL4 (BioBricks reference BBA_k511003; FIG. 8B) test promoter is cloned upstream of the Red Fluorescent Protein Generator (mKATE) (BioBricks reference BBA_k511102; FIG. 7B), and this construct is cloned upstream of the reporter construct Hef1a-ERFP2 (BioBricks reference BBA_k511800; FIG. 6).

(iii) a UAS-GAL4 (BioBricks reference BBA_k511003; FIG. 8B) test promoter is cloned upstream of the Blue fluorescent protein reporter EBFP2 (BioBricks reference BBA_k511100; FIG. 7A), and this construct is cloned upstream of the reporter construct Hef1a-mKATE (BioBricks reference BBA_k511 801; FIG. 5).

(iv) a TRE-Tight-LacOid promoter (BioBricks reference BBA_k511004; FIG. 9) test promoter is cloned upstream of the Red Fluorescent Protein Generator (mKATE) (BioBricks reference BBA_k511102; FIG. 7B), and this construct is cloned upstream of the reporter construct Hef1a-EBFP2 (BioBricks reference BBA_k511 800; FIG. 6).

(v) a TRE-Tight-LacOid promoter (BioBricks reference BBA_k511004; FIG. 9) test promoter is cloned upstream of the Blue fluorescent protein reporter EBFP2 (BioBricks reference BBA_k511100; FIG. 7A), and this construct is cloned upstream of the reporter construct Hef1a-mKATE (BioBricks reference BBA_k511801; FIG. 5).

Other embodiments are provided in the claims.

What is claimed is:

1. A method of evaluating a candidate genetic element comprising:
(i) providing a cell comprising a nucleic acid molecule comprising a test genetic element that controls expression of a first reporter gene, and a control genetic element that controls expression of a second reporter gene;
(ii) incubating the cell for a time and under conditions sufficient to allow expression of the test genetic element;
(iii) measuring the relation of expression of the first reporter gene to the second reporter gene,
thereby evaluating the strength of the candidate genetic element.

2. The method of claim 1, wherein the step of providing a cell comprising a nucleic acid molecule comprising a test genetic element that controls expression of a first reporter protein, and a control genetic element that controls expression of a second reporter promoter comprises:
(i) providing the nucleic acid molecule comprising a test genetic element that controls expression of a first reporter gene, and a control genetic element that controls expression of a second reporter gene, and
(ii) transfecting the nucleic acid molecule into the cell,
thereby providing a cell comprising a nucleic acid molecule comprising a test genetic element that controls expression of a first reporter gene, and a control genetic element that controls expression of a second reporter gene.

3. The method of claim 1, wherein the genetic element is a DNA promoter.

4. The method of claim 1, wherein the control genetic element is a constitutive promoter.

5. The method of claim 1, wherein the test genetic element is an inducible promoter, an activatable promoter, a repressible promoter or a hybrid promoter.

6. The method of claim 1, wherein the control genetic element is an inducible promoter.

7. The method of claim 1, wherein the first reporter gene and the second reporter gene encode fluorescent proteins.

8. The method of claim 1, wherein the first reporter gene encodes Enhanced Blue Fluorescent Protein 2 (EBFP2), mKate, infrared fluorescent protein (iRFP) or enhanced yellow fluorescent protein (EYFP).

9. The method of claim 8, wherein the second reporter gene encodes EBFP2, mKate, iRFP or EYFP, and wherein the second reporter gene is different than the first reporter gene.

10. The method of claim 9, wherein the first reporter gene and the second reporter gene are in a mammalian cell.

11. The method of claim 1, wherein the first reporter gene encodes Green Fluorescent Protein (GFP), Red Fluorescent Protein (RFP) or Blue Fluorescent Protein (BFP).

12. The method of claim 11, wherein the second reporter gene encodes GFP, RFP or BFP, and wherein the second reporter gene is different than the first reporter gene.

13. The method of claim 12, wherein the first reporter gene and the second reporter gene are in a prokaryotic cell.

14. The method of claim 12, wherein the first reporter gene and the second reporter gene are in an E. coli cell.

15. The method of claim 1, wherein the test genetic element is identical to an element that is in a circuit.

16. The method of claim 1, wherein the nucleic acid is a plasmid.

17. The method of claim 16, wherein the plasmid does not replicate in mammalian cells.

18. The method of claim 7, wherein the test genetic element controlling expression of the first fluorescent protein and the control genetic element controlling expression of the second fluorescent protein are positioned in the same orientation on the nucleic acid.

19. The method of claim 7, wherein the cell comprises only one copy of the nucleic acid comprising the test genetic element that controls expression of the first fluorescent protein, and a control genetic element that controls expression of the second fluorescent protein.

20. The method of claim 7, wherein the cell comprises multiple copies of the nucleic acid comprising the test genetic element that controls expression of the first fluorescent protein, and the control genetic element that controls expression of the second fluorescent protein.

21. The method of claim 1, wherein the cell is a mammalian cell.

22. The method of claim 1, wherein the cell is a prokaryotic cell.

23. The method of claim 1, wherein the cell is an E. coli cell.

24. The method of claim 1, wherein the test genetic element is a member of a library.

25. A circuit comprising a nucleic acid molecule comprising a test genetic element that controls expression of a first reporter gene, and a control genetic element that controls expression of a second reporter gene.

26. The circuit of claim 25, wherein the test genetic element and the control genetic element are each a DNA promoter.

27. The circuit of claim 25, wherein the first and second reporter genes encode fluorescent proteins.

28. A cell comprising a nucleic acid molecule comprising a test genetic element that controls expression of a first reporter gene and a control genetic element that controls expression of a second reporter gene.

29. The cell of claim 28, wherein the test genetic element and the control genetic element are each a DNA promoter.

30. The cell of claim 28, wherein the first and the second reporter genes encode fluorescent proteins.

31. A method of identifying a candidate genetic element for use in a circuit, comprising:
(i) providing a plurality of test genetic elements, wherein each test genetic element of the plurality is provided in a separate test nucleic acid molecule for control of a first reporter gene, and each test nucleic acid molecule is operably linked to a control genetic element that controls expression of a second reporter gene;
(ii) expressing one or more of the test nucleic acid molecules in a cell for a time and under conditions sufficient to allow expression from the test genetic element and the control genetic element;
(iii) measuring the relation of expression of the first reporter gene and the second reporter gene, wherein if the relation is determined to be at a desired level for use in the circuit, then the test genetic element is chosen for use in the circuit,
so that the candidate genetic element is identified for use in a circuit.

32. The method of claim 31, wherein the test genetic element and the control genetic element are each a DNA promoter.

33. The method of claim 31, wherein the first and the second reporter genes encode fluorescent proteins.

34. A library comprising a plurality of test genetic elements wherein each test genetic element of the plurality is positioned 5′ to a first reporter gene, wherein each first reporter gene is positioned 5′ to a control genetic element that is positioned 5′ to a second control reporter gene.

35. The library of claim 34, wherein the genetic elements are DNA promoter elements.

36. The library of claim 34, wherein each test genetic element is flanked on either side by a means for cloning.

37. The library of claim 36, wherein the means for cloning comprises restriction enzyme recognition sites or sequences for recombination cloning.

38. The library of claim 34, wherein the test genetic element and the control genetic element are each a DNA promoter.

39. The library of claim 34, wherein the first reporter gene and the second reporter gene encode a first and second reporter protein, respectively.

40. A method of making a library comprising a plurality of test genetic elements, wherein the method comprises introducing each test genetic element of the plurality into a plasmid, wherein each test genetic element of the plurality is positioned 5′ to a first reporter gene, wherein each first reporter gene is positioned 5′ to a control genetic element that is positioned 5′ to a second control reporter gene, and wherein each test genetic element is introduced into the plasmid by a restriction enzyme based method or by recombination cloning method.

41. The method of claim 40, wherein the test genetic element and the control genetic element are DNA promoter elements.

42. The method of claim 40, wherein the first reporter gene and the second reporter gene encode a first and second reporter protein, respectively.

43. The method of claim 1, 25, 28 or 31 wherein the test genetic element and the control genetic element are coupled on the same nucleic acid molecule.

44. The method of claim 1, 25, 28 or 31 wherein the test genetic element and the control genetic element are coupled on different nucleic acid molecules.

45. The method of claim 1, 25, 28 or 31 wherein the first reporter gene encodes a fluorescent protein, a chemiluminescent protein, a protein that can be detected by immunostaining, or a protein that can be radioactively labeled.

46. The method of claim 1, 25, 28 or 31 wherein the second reporter gene encodes a fluorescent protein, a chemiluminescent protein, a protein that can be detected by immunostaining, or a protein that can be radioactively labeled.
